(19) United States
(12) Patent Application Publication
(10) Pub. No.: US 2013/0261554 A1
(43) Pub. Date: Oct. 3, 2013

(54) INTRAVENOUS CATHETER APPARATUS

(71) Applicant: Rishi Baid, New Delhi (IN)

(72) Inventor: Rishi Baid, New Delhi (IN)

(73) Assignee: POLY MEDICURE LIMITED, Faridabad, Haryana (IN)

(21) Appl. No.: 13/991,768

(22) PCT Filed: Feb. 14, 2011
(86) PCT No.: PCT/IB11/50596

§ 371 Date: Jun. 5, 2013

(30) Foreign Application Priority Data
Dec. 6, 2010 (IN) 2897/DEL/2010

(51) Int. Cl. A61M 25/06

(52) U.S. Cl. CPC A61M 25/0606
USPC 604/164.06

(57) ABSTRACT

The invention relates to an intravenous catheter apparatus comprising a tubular catheter having a proximal end and a distal end, a needle defining an axial direction and having a needle shaft and a needle tip at a distal end of the needle shaft, wherein said needle shaft extends through said tubular catheter such that said needle tip of said needle protrudes from said distal end of said tubular catheter, and wherein said needle shaft comprises an engagement means adapted to engage with a needle guard slidably arranged on said needle shaft in order to prevent said needle guard from sliding off said needle tip.

The invention relates to an intravenous catheter apparatus comprising a tubular catheter having a proximal end and a distal end, a needle defining an axial direction and having a needle shaft and a needle tip at a distal end of the needle shaft, wherein the needle shaft extends through the catheter such that the needle tip protrudes from the distal end of the tubular catheter, and wherein the needle shaft comprises an engagement means adapted to engage with a needle guard slidably arranged on the needle shaft in order to prevent the needle guard from sliding off the needle tip.

An intravenous catheter apparatus of this kind is generally known and, for example, used to administer medicine to a patient or to take blood from a patient.

In use, the tubular catheter is inserted into a blood vessel of the patient, typically a vein, by means of the needle which will be withdrawn from the catheter after insertion of the tubular catheter into the blood vessel. When the needle tip enters the blood vessel, blood flows through a lumen of the needle into a needle hub provided at a proximal end of the needle, where it can be observed by the person handling the catheter apparatus. The event of blood entering the needle hub is called blood flashback and is used by the person handling the catheter apparatus to confirm venipuncture, from which point on the person inserts the catheter as far as desired under a decreased inclination of the needle in order to not puncture the back wall of the blood vessel.

In the case of delayed blood flashback there is a risk that the person handling the catheter apparatus continues to push the needle into the blood vessel under the increased original inclination of the needle unit it exits at the rear side of the blood vessel. This second venipuncture represents a risk to the health of the patient as it may result in inner bleeding. Apart from that the catheter apparatus will have to be removed and replaced by a new one, i.e. the process of introducing the catheter will have to be repeated which is unpleasant and painful to the patient.

It is an object of the invention to provide an intravenous catheter apparatus which allows for safer and more reliable placement of the catheter in the patient's blood vessel.

This object is satisfied by an intravenous catheter apparatus in accordance with claim 1.

The intravenous catheter apparatus of the invention comprises a tubular catheter having a proximal end and a distal end. A needle defines an axial direction and has a needle shaft and a needle tip at a distal end of the needle shaft, wherein the needle shaft extends through the tubular catheter such that the needle tip protrudes from the distal end of the tubular catheter. The needle shaft comprises an engagement means adapted to engage with a needle guard slidably arranged on the needle shaft in order to prevent the needle guard from sliding off the needle tip. The needle shaft further comprises a lateral opening arranged between the engagement means and the needle tip such that the opening is covered by the tubular catheter.

The lateral opening provides communication between a lumen of the needle and an interior of the tubular catheter. In the event of first venipuncture blood entering the lumen of the needle can exit the needle through the lateral opening and thus become visible for the person handling.

Because of the position of the lateral opening close to the needle tip, the blood does not have to travel the length of the needle to enter the needle hub in order to become visible. Instead, blood entering the lumen of the needle upon venipuncture partly exits the needle again near the needle tip, thereby becoming particularly quickly and, thus, allowing for particularly fast venipuncture confirmation.

In addition, because of the arrangement of the lateral opening between the engagement means and the needle tip, the blood does not have to pass the engagement means to become visible in the needle hub. Hence, the time at which blood entering the needle upon venipuncture becomes visible to the person handing the catheter apparatus, is independent of the design of the engagement means. Hence, even if the engagement means is designed such that it would normally slow down the flow of blood through the needle and, thus, delay blood flashback, it does not have any adverse effect on the timing of venipuncture confirmation in the apparatus of the invention.

As a result, the intravenous catheter apparatus of the invention allows faster and more reliable confirmation of successful venipuncture which makes the handling of the catheter apparatus easier both for the person placing it and the patient on whom it is used.

In order to improve the visibility of the blood exiting the needle through the lateral opening, the tubular catheter advantageously comprises a transparent material.

According to a preferred embodiment, the lateral opening comprises a slot cut into the needle shaft in a direction transverse to the axial direction. Such a slot is easy to manufacture and thus helps to minimize manufacturing costs of the catheter apparatus.

In order to enhance spreading of the blood exiting the needle through the lateral opening inside the tubular catheter, an outer diameter of the needle shaft may be slightly smaller than an inner diameter of the tubular catheter. The enhanced spreading of the blood improves visibility of the blood through the tubular catheter and, thus, makes detection of successful venipuncture faster and more reliable.

In order to make the insertion of the tubular catheter into a patient's blood vessel even more agreeable, the tubular catheter may slightly tapered in its distal end region such that the distal end of the tubular catheter tightly surrounds the needle shaft.

According to a further embodiment the engagement means comprises an enlargement of the needle shaft in at least one direction transverse to the axial direction.

An engagement means that is particularly easy to manufacture comprises a crimped portion of the needle shaft. The manufacturing costs of the needle thus can be further reduced.

A preferred embodiment of the invention is described in the following description and in the accompanying drawings, wherein:

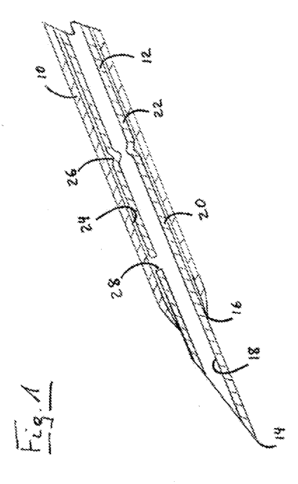

FIG. 1 shows a longitudinal sectional view of a distal end region of an intravenous catheter apparatus in accordance with the invention.

FIG. 1 shows a distal end portion of a tubular catheter 10 of an intravenous catheter apparatus of the invention. The tubular catheter 10 is made of a transparent material, for example, a transparent plastic material.

A needle 12 defining an axial direction extends through the tubular catheter 10 such that a distal tip of the needle 12 protrudes from a distal end 16 of the tubular catheter 10. The needle 12 has a lumen 18 which extends along the length of the needle 12 in the axial direction and is defined by a wall 20 of the needle 12. The needle wall 20 forms a needle shaft 22.

The outer diameter of the needle shaft 22 is slightly smaller than the inner diameter of the tubular catheter 10 in order to provide a small gap 24 between the needle shaft 22. In its distal end region of the tubular catheter 10 is slightly tapered such that the distal end 16 of the tubular catheter 10 tightly surrounds the needle shaft 22.

Proximal from the needle tip 14 the needle shaft 22 is provided with an engagement means 26 for preventing a needle guard (not shown) slidably arranged on the needle shaft 22 from moving beyond the needle tip 14. The engagement means 26 may comprise any form of irregularity of the needle shaft 22, for example, an enlargement of the outer profile of the needle shaft 22 at least in one direction transverse to the axial direction.

In the illustrated embodiment, the engagement means 26 comprises a crimped portion of the needle shaft 22.

The needle guard may comprise a generally cylindrical base portion made of a plastic material and having an axial bore through which the needle 12 extends. The inner diameter of the bore is adapted to the principal outer diameter of the needle shaft 22 such that the needle shaft 22 is free to slide through the bore, but the engagement means 26 of the needle 12 cannot pass the base portion of the needle guard.

The needle guard may further comprise two arms provided at the distal side of the base portion and extending generally in the axial direction therefrom. At least one of the arms has elastic properties such that it can be deflected slightly off the axial direction by the needle shaft 22. Both of the arms may be integrally formed with the base portion. Alternatively, one of the arms may be integral with the base portion whereas the other one of the arms may be made of a strip of sheet metal.

The length of at least one elastic arm is longer than the distance between the needle tip 14 and the engagement means 26, such that a bent distal end section of the arm can move in front of the needle tip 14 and capture the needle tip 14 in the needle guard before the engagement means 26 of the needle 12 engages with the base portion of the needle guard.

Preferably, the arms are surrounded by an elastic band which biases the arms towards the needle 12. The elastic band may cover a substantial portion of the arms seen in the axial direction and, thus, prevent the needle tip 14 captured between the arms from protruding sideway out of the needle guard.

A lateral opening 28 is provided in the needle wall 20 in a region between the needle tip 14 and the engagement means 26. The lateral opening 28 is positioned such that it is covered by the tubular catheter 10 when the needle tip 14 protrudes from the distal end 16 of the tubular catheter 10, i.e. it is arranged between the engagement means 26 and the distal end 16 of the tubular catheter 10. The lateral opening 28 thus provides communication between the lumen 18 of the needle 12 and the interior of the catheter.

When the needle 12 together with the tubular catheter 10 is inserted into a blood vessel of a patient, blood flows into the lumen 18 of the needle 12 in the event of venipuncture. The blood flowing into the lumen 18 of the needle 12 partly exits the needle 12 through the lateral opening 28 and enters the gap 24 between the needle shaft 22 and the tubular catheter 10. The blood entering the gap 24 between the needle shaft 22 and the tubular catheter 10 spreads in the gap 24 due to capillary action and can be seen through the transparent material of the tubular catheter 10, thereby allowing prompt confirmation of successful venipuncture.

### REFERENCE NUMERALS

10 tubular catheter
12 needle
14 needle tip
16 distal end
18 lumen
20 needle wall
22 needle shaft
24 gap
26 engagement means
28 lateral opening

1. An intravenous catheter apparatus comprising:
a tubular catheter (10) having a proximal end and a distal end (16);
a needle (12) defining an axial direction and having a needle shaft (22) and a needle tip (14) at a distal end of the needle shaft (22);
said needle shaft (22) extending through said tubular catheter (10) such that said needle tip (14) of said needle (12) protrudes from said distal end (16) of said tubular catheter (10); and
said needle shaft (22) comprising an engagement means (26) adapted to engage with a needle guard slidably arranged on said needle shaft (22) in order to prevent said needle guard from sliding off said needle tip (14),
characterized in that
said needle shaft (22) further comprises a lateral opening (28) arranged between said engagement means (26) and said needle tip (14) such that said lateral opening (28) is covered by said tubular catheter (10).

2. An intravenous catheter apparatus in accordance with claim 1,
characterized in that
said lateral opening (28) provides communication between a lumen (18) of said needle (12) and an interior of said tubular catheter (10).

3. An intravenous catheter apparatus in accordance with claim 1,
characterized in that
said lateral opening (28) comprises a slot cut into said needle shaft (22) in a direction transverse to said axial direction.

4. An intravenous catheter apparatus in accordance with claim 1,
characterized in that
an outer diameter of the needle shaft (22) is slightly smaller than an inner diameter of said tubular catheter (10).

5. An intravenous catheter apparatus in accordance with claim 1,
characterized in that
said tubular catheter (10) is slightly tapered in its distal end region such that the distal end (16) of the tubular catheter (10) tightly surrounds the needle shaft (22).

6. An intravenous catheter apparatus in accordance with claim 1,
characterized in that
said engagement means (26) comprises an enlargement of said needle shaft (22) in at least one direction transverse to the axial direction.

7. An intravenous catheter apparatus in accordance with claim 1,
characterized in that
said engagement means (26) comprises a crimped portion of said needle shaft (22).

8. An intravenous catheter apparatus in accordance with claim 1,
characterized in that
said tubular catheter (10) comprises a transparent material.
